(19) United States
(12) Patent Application Publication
(10) Pub. No.: US 2014/0098342 A1
(43) Pub. Date: Apr. 10, 2014

(54) SYSTEM AND METHOD FOR CORNEAL IRRADIATION

(71) Applicant: The General Hospital Corporation, (US)

(72) Inventor: Robert H. Webb, Lincoln, MA (US)

(73) Assignee: The General Hospital Corporation, Boston, MA (US)

(21) Appl. No.: 13/666,950

(22) Filed: Nov. 1, 2012

Related U.S. Application Data
(60) Provisional application No. 61/555,520, filed on Nov. 4, 2011.
(60) Provisional application No. 61/603,482, filed on Feb. 27, 2012.

(51) Int. Cl. A61B 3/00; A61B 3/12

(52) U.S. Cl. CPC A61B 3/0008; A61B 3/12
USPC 351/206; 351/221; 351/246

(57) ABSTRACT

A device and method for use thereof to illuminate a visual system of a subject includes a light-transforming optical element configured to transform a substantially collimated beam of light into light having a diverging spatial distribution. Optionally, light having such spatial distribution includes a plurality of diverging beams of light. An imaging system mechanically cooperated with the light-transforming optical element is configured such as to form an image of the light-transforming optical element at an image surface associated with the eye that is distant from the retina. The irradiance level at the image surface exceeds that at the retina. Optionally, the image surface adjoins or includes the cornea. The imaging system may include an optical system containing refractive and/or reflective optical elements.

### CROSS-REFERENCE TO RELATED APPLICATIONS

The present application claims priority from and benefit of the U.S. Provisional Patent Applications No. 61/555,520 titled “Method and Apparatus for Corneal Irradiation” filed on Nov. 4, 2011 and 61/603,482 titled “System and Method for Corneal Irradiation” filed on Feb. 27, 2012. The disclosure of each of the above-mentioned provisional applications is incorporated herein by reference in its entirety for all purposes.

### STATEMENT REGARDING FEDERALLY SPONSORED RESEARCH OR DEVELOPMENT

This invention was made with government support under Grant Number W81XWH-09-2-0050 awarded by the Department of Defense. The government has certain rights in the invention.

### TECHNICAL FIELD

The present invention relates to delivery of light to a subject's visual system and, more particularly, to irradiation of the cornea at levels that are safe for the retina.

### BACKGROUND ART

Examination and optional treatment of a condition of a visual system (of a human, for example) often utilizes light, for obvious reasons that at least an eye-portion of the visual system partially transmits light. Light therapy or phototherapy may include an exposure of a biological target such as an eye, for example, to daylight or to specific wavelengths of light (using artificial sources such as lasers, light-emitting diodes, fluorescent lamps, dichroic lamps or very bright, full-spectrum light, usually controlled with various devices), and also to facilitate visualization or other detection of defects of the visual system, and to catalyze and/or promote certain physicochemical reactions in the visual system. The light is administered for a prescribed amount of time and, in some cases, at a specific time of day. Light therapy of the retina of an eye, for example, is used to treat circadian rhythm disorders such as delayed sleep phase syndrome and can also be used to treat seasonal affective disorders, with some support for its use also with non-seasonal psychiatric disorders.

Similarly, irradiation of an ocular surface can, under certain conditions, facilitate a repair of ocular surface defects, closure of an incision or attachment of a graft tissue with sutures or a biologically-compatible adhesive. Conventionally, the ocular surface is viewed to include the cornea and its major support tissue, the conjunctiva. While in a wider anatomical and also functional sense, the ocular mucosal adnexa (i.e. the lacrimal gland and the lacrimal drainage system) also belongs to the ocular surface, it is the cornea that is directly exposed to the external environment, and therefore is endangered by a multitude of antigens, pathogenic microorganisms, and mechanical influences. Repair of a corneal surface has been demonstrated, for example, with the use a so-called Photochemical Tissue Bonding (PTB) involving a light-activated bonding of corneal tissues or cross-linking of corneal proteins. In this example, the PTB may be facilitated with a light-activated agent such as a Rose Bengal, and performed at irradiance levels in a range of approximately 0.2-1.0 W/cm² or so, with typical fluence values on the order of about 50 J/cm² to about 200 J/cm². An example of the PTB-based technique used for bonding of an amniotic membrane to an ocular surface for repairing ocular surface defects is provided by Wang et al. in “Lasers in Surgery and Medicine” (43:481-489, 2011)

Corneal irradiation with a substantially collimated beam of light causes at least a portion of such beam that traverses the cornea be focused at the retinal surface and, under some conditions, exceed the Maximum Permissible Exposure (MPE) delineated in ANSI A136.1-2007. (An overview of factors expressed in this standard and rationale behind the use of these factors is provided, for example, by Delori et al. in J. Opt. Soc, Amer. A, 24(5), 1250-1265, 2007.)

Accordingly, there is a need in an apparatus and method for irradiation of an ocular surface and, in particular, the cornea at such spatial distribution of light that ensures levels of exposure that are sufficiently high to effect therapy-enhancing reactions and, at the same time, below a threshold defined by optical damage to the retina.

### SUMMARY OF THE INVENTION

Embodiments of the present invention provide a device for illumination of a visual system of a subject. Such device includes an optical transformer element configured to transform a substantially collimated beam of light into a spatially diverging distribution of light (half-angle of divergence, in one case, is between about 10 and about 20 degrees) and an imaging system mechanically cooperated with said optical transformer element. The spatially diverging distribution of light optionally includes a multiplicity of diverging beams of light. The imaging system is adapted to transmit a portion of said spatially diverging distribution of light onto an eye of the subject and to form an image of said optical transformer element at an image surface defined not to coincide with the retinal surface of the subject. Such imaging surface is optionally defined at an ocular surface of the subject and, in a specific implementation, at a surface of the cornea. The imaging system may include a lens and/or a mirror, and, in one embodiment, is structured as a telecentric system.

The optical transformer may include a holographically-defined light-diffusing element. In a related implementation, the optical transformer includes a diffractive element that transforms a substantially collimated beam, incident on such diffractive element, into optical point sources (optionally, an array of spots of light or disconnected spots of light) in far field. In a related embodiment, the optical transformer includes at least one of an array of apertures and an array of optical lenslets. A specific example of the array of optical lenslets includes lenslets that adjoin each other along their corresponding non-circular perimeters. A surface defined by the optical transformer is generally curved, but in a specific embodiment may be planar.

The device optionally further includes a fiber-optic (FO) element having an input facet adapted to receive light from a source of light and an output facet in optical communication with the optical transformer element such as to deliver a substantially collimated beam of light to the optical transformer element. In addition or alternatively, the device can include a masking element operably cooperated with a component of the device such as to block a portion of light that forms the image of the optical transformer element at the image surface. In a specific implementation, the masking element is positioned proximate to at least one of i) a location of an optical conjugate of an ocular surface and ii) a location of an optical conjugate of the retinal surface.

Embodiments of the invention further provide an optical relay system for illumination of a visual system of a subject, which optical relay system includes i) an optical diffuser configured to transform a beam of light incident onto the optical diffuser to light having a diverging spatial distribution and ii) an optical system configured to form an image of the optical diffuser at an image surface, which is defined substantially at a corneal surface of the subject when the optical system is positioned for transmitting light from the optical diffuser through the optical system to irradiate the visual system of the subject. The optical diffuser generally reflects or transmits light in a spatially diffusive fashion. In particular, the optical diffuser includes at least one of a translucent holographically-defined optical diffuser and a diffractive optical element configured to receive a substantially collimated beam of light and form from such collimated beam of light a far-field light distribution that defines an array of spatially-disconnected spots of light. The optical system is substantially co-axial with a normal to a surface of the optical diffuser and is enabled to form an image of the optical diffuser in light transmitted through the optical diffuser. Alternatively or in addition, the optical relay system optionally includes a fiber-optic (FO) element having an input facet adapted to receive light from a source of light and an output facet that is optically cooperated with the optical diffuser such as to deliver a substantially collimated beam of light to the optical diffuser. Furthermore, the optical relay system optionally includes a masking element operably cooperated with a component of the device and configured to block a portion of light that forms the image of the optical diffuser. When present, such masking element is positioned proximate to at least one of i) a location of an optical conjugate of the corneal surface as defined by the optical system and ii) a location of an optical conjugate of a retinal surface defined in front of the corneal surface. The optical system optionally includes a telecentric optical system.

Embodiments of the invention further provide an optical relay system for illumination of a visual system of a subject. Such optical relay system includes a light diffusing component—an optical diffuser configured to transform a beam of light incident onto the optical diffuser, by diffusely reflecting or diffusely transmitting such incident light, to light having a diverging spatial distribution. The optical diffuser has a surface defining a normal to the surface. An optical system is structured to irradiate a corneal surface of the subject when the optical system is positioned for transmitting light from the optical diffuser through the optical system to irradiate the retina. Such irradiation is characterized by an irradiance value, at the corneal surface of the subject, that exceeds an irradiance value at a retinal surface of the subject.

Moreover, embodiments of the invention provide a method for illuminating an ocular surface of an eye of a subject. The method includes receiving light from an optical transformer element adapted to transform a substantially collimated beam of light into light having a diverging spatial distribution; and imaging the optical transformer element with an optical imaging system onto an image surface in front of or behind a retina of the eye such as to transmit a portion of light received from the optical transformer element through a cornea of the eye. In one embodiment, the optical transformer element is configured to form, in transmission, a spatial distribution of light having an angle of divergence between about 10 and 20 degrees. In a related embodiment, the optical transformer element includes an array of optical apertures and, accordingly, receiving light from an optical transformer element includes receiving light that has traversed an array of optical apertures. In a specific related embodiment, light received from the optical transformer element has traversed an array of optical apertures (optionally—apertures with non-zero optical power including first and second apertures that have non-circular perimeters and that adjoin each other along such non-circular perimeters. In yet another case, the optical transformer element is configured to receive a substantially collimated beam of light and to form, from such collimated beam of light, a far-field light distribution including an array of point sources of light.

Imaging of the optical transformer onto an image surface includes, for example, imaging of the optical transformer element with an optical imaging system having a magnification that is independent from a distance between a principal plane of the optical imaging system and the optical transformer element. In one embodiment, such imaging is performed with a telecentric imaging system.

Furthermore, the method of the invention optionally includes, in addition, at least one of receiving a substantially collimated beam of light at the optical transformer element and transmitting light through the optical transformer element towards the imaging optical system such as a telecentric system. In addition or alternatively, the method optionally utilizes delivering a substantially collimated beam of light to the optical transformer element from a source of light through a fiber-optic (FO) element and/or blocking a portion of light, which light defines the image of the optical transformer element, between the optical transformer element and the cornea. For example, blocking a portion of light may be effectuated by placing a masking element proximate to a location of an optical conjugate of the cornea.

### BRIEF DESCRIPTION OF THE DRAWINGS

The invention is more fully understood by referring to the following Detailed Description in conjunction with the Drawings, of which:

FIG. 1A is a diagram depicting a conventional system employed for irradiation of the cornea.

FIG. 1B is a diagram illustrating an idea of the present invention.

FIG. 2 is a diagram of the system according to an embodiment of the invention.

FIG. 3A is a schematic layout of an optical transformer element for use with an embodiment of the invention.

FIG. 3B is a schematic layout of an alternative implementation of the optical transformer element for use with an embodiment of the invention.

FIGS. 3C and 3D are front and side views of another implementation of the optical transformer element for use with an embodiment of the invention.

FIG. 4 is a diagram of the system according to an alternative embodiment of the system of the invention.

FIGS. 5A and 5B illustrate alternative embodiments of the system of the invention.

### DETAILED DESCRIPTION

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

In addition, the following disclosure may describe features of the invention with reference to corresponding drawings, in which like numbers represent the same or similar elements wherever possible. In the drawings, the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed.

The invention as recited in claims appended to this disclosure is intended to be assessed in light of the disclosure as a whole.

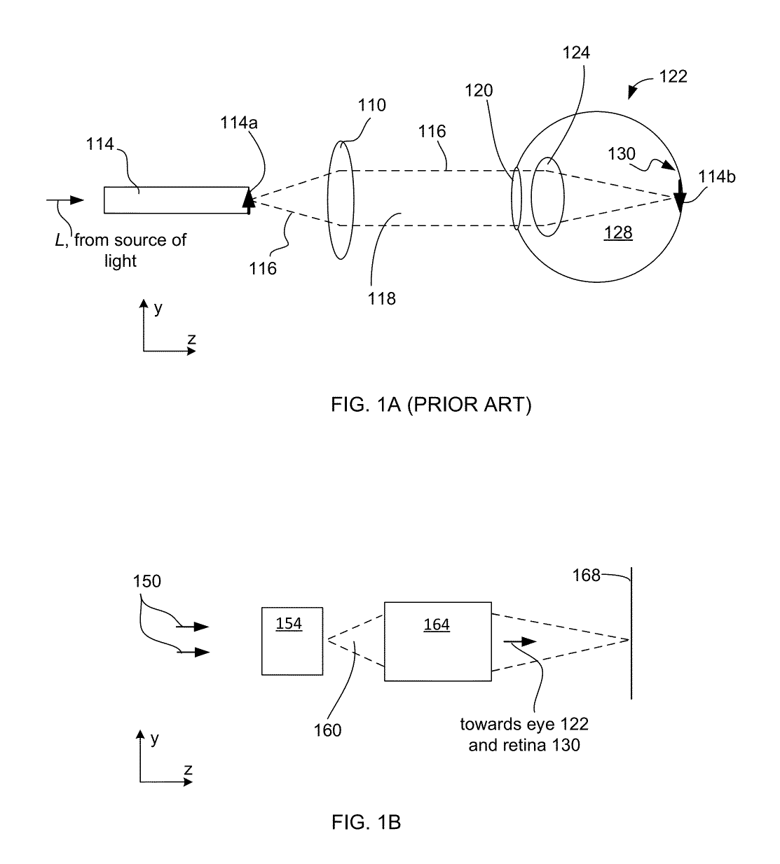

Conventionally, targeted irradiation of an ocular surface (such as the corneal surface, for example) is carried out with light incident onto the ocular surface either directly from a distant source of light or through an optical system (that delivers a substantially collimated beam to the ocular surface, for instance). An example of such situation is schematically illustrated in the diagram of FIG. 1A. Light L from an external source of light (not shown) is transmitted towards an optical collimator 110 through an optical fiber 114, an output facet of which is represented by an arrow 114a and functions as a local source of light. The following propagation of light is schematically shown with the dashed lines 116. Upon traversing the collimator 110, light is substantially collimated and impinged as a beam 118 upon a corneal surface 120 of an eye 122. The incident light 118 further passes through the anterior chamber and pupil (not shown), and is converged by the lens 124 through the vitreous humour 128 onto the retina 130. Clearly, as long as light 118 incident onto the ocular surface (such as the corneal surface 120) is substantially collimated, an image 114b of the local source of light 114a is formed at or very close to the retinal surface, thereby irradiating the retinal surface 130 at power levels that may exceed the MPE determined thresholds. If, for example, a 20× objective is used as the lens 110, the corneal surface 120 of about 12 mm in diameter may be irradiated over an area of about 400 times larger than that of the fiber's output facet. A typical dimension of the image 114b at the retina 130 in this situation is about 1.4 mm in diameter, thereby resulting in the irradiance level at the retina 130 of about 75-fold that at the cornea 120. In another conventionally used arrangement and in further reference to FIG. 1A, light from an LED source, for example, can be collected by the lens 110 and further relayed towards the eye 122. A conventional system and method of irradiating the corneal surface, therefore, may lead to damaging the retinal surface.

Implementations of the present invention address this shortcoming and provide an optical system and method that facilitate the irradiation of the ocular surface of the eye (for example, its corneal surface) at desired light-density levels while, at the same time, ensuring that light irradiance at the retinal surface remains below established ophthalmological thresholds. A diagram of FIG. 1B illustrates the general idea of the invention. A substantially spatially collimated distribution of light 150 is delivered to and received by an optical transformer element 154, configured to convert such beam 150 into light 160 having spatially-diverging distribution. Light 160 is passed on to an imaging system 164 and, upon traversing the system 164, towards the eye 122 that is accordingly illuminated and traversed by the incident light. The imaging system 154 is adapted to form an image of the element 154 at a surface 168 located at a distance from (either in front of or behind) the retina 130. More specifically, embodiments of the present invention are configured to ensure that light irradiance at the cornea is higher than light irradiance at the retina.

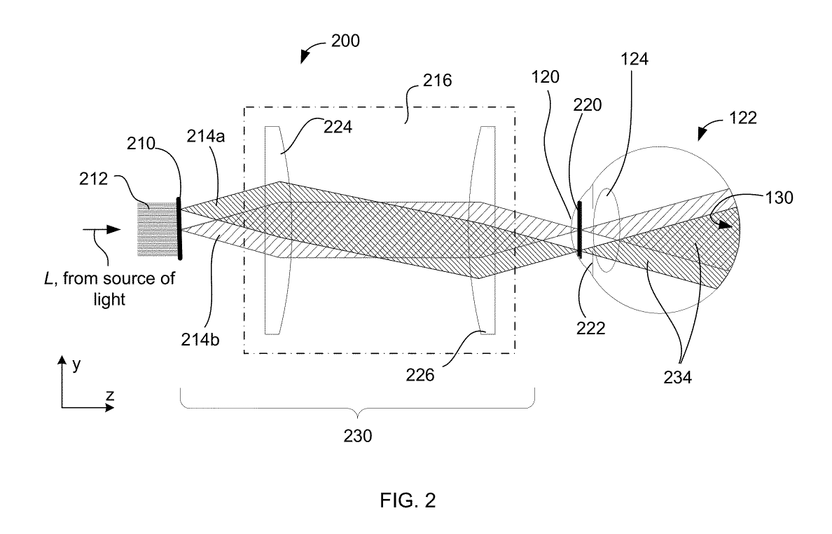

To this end, as shown in the example of a diagraph of FIG. 2, an implementation 200 of a device configured for illumination of a visual system of a subject employs an optical transformer element 210 structured to transform an optionally substantially collimated beam 212 of light received from a source of light (not shown) into light having, generally, a diverging spatial distribution. In this specific example, such light passing through the element 220 includes a plurality of diverging beams (as shown, two beams 214a, 214b). The embodiment 200 further includes an optical system 216 that is, generally, mechanically cooperated with the optical element 210 and adapted to form an image 220 of the optical transformer element 210 at an image surface associated with the eye 122 and that is located away from the retinal surface 130. In the specific case illustrated in FIG. 2 such image surface 220 is located within the eye 122 in front of the retinal surface 130 and is distanced from the retinal surface 130. In another specific case (not shown), the surface at which the image of the optical transformer element 210 is formed by the optical system 216 is located behind the retinal surface as viewed from the cornea 120.

It is appreciated, that imaging of the optical transformer element 210 onto a surface such as the image surface 220 of FIG. 2 does not have to be precise. For example, an optical imaging system such as the imaging system 216 of FIG. 2 may have residual optical aberrations, or be misaligned, or otherwise configured such that the image of the optical transformer element 210 is, for example, somewhat blurred. An embodiment of the invention is advantageous over conventionally-utilized imaging systems in that the quality of its operation is not sufficiently affected by imperfections of the optical imaging system of the embodiment.

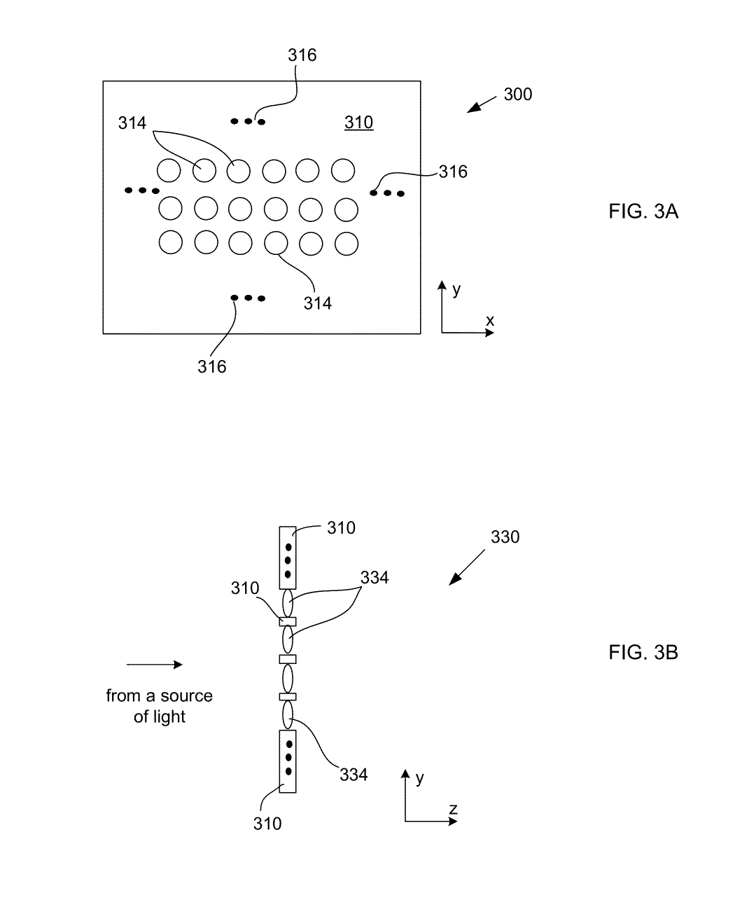

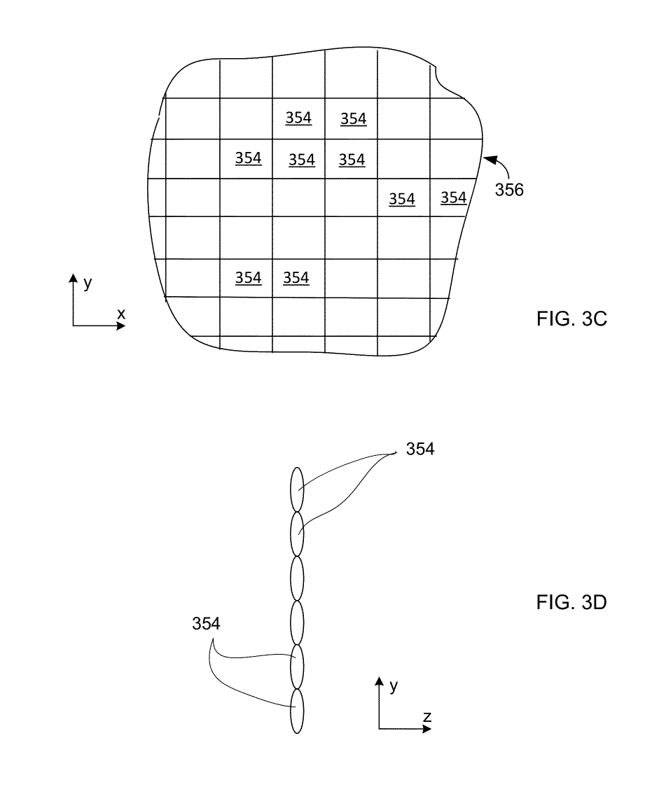

In one example, the optical element 210 includes a plurality of apertures (in a screen that may be otherwise substantially opaque or translucent at the wavelength(s) of interest), optionally arranged as a one-dimensional or two-dimensional arrays of apertures. Passing through such plurality of apertures light 212 diffracts to form the diverging beam(s) such as the beams 214a, 214b. An example 300 of the optical element 210, containing a screen 310 (whether opaque or translucent) with a two-dimensional (2D) array of light-transmitting apertures 314, is schematically shown in FIG. 3A. The ellipses 316 in FIG. 3A represent an optional repetition of a spatial pattern formed by the apertures 314. The dimensions and/or shapes of the apertures 314 are appropriately defined, with respect to the spectral content and degree of collimation of the incident light 212, to ensure that angles of divergence of the plurality of beams such as beams 214a, 214b of FIG. 2 do not exceed a pre-determined value. In one implementation, the angles of divergence of the beams 214a, 214b (measured as half-a-linear angle corresponding to the numerical aperture of the beams 214a, 214b), are between about 10 and about 40 degrees, preferably within the range of about 10 to about 20 degrees, more preferably about 13 to about 17 degrees, and even more preferably about 15 degrees. The term “about”, as used in this application with respect to a stated value of measure, defines a deviation from such stated value that is typical for usage of the stated measure in the art. The precise shape and degree of being opaque of the screen 310 does not change the principle of operation of the optical element 210. Similarly, a spatial pattern formed by and the number of the apertures 314 do not change the principle of operation of the invention. In one specific non-limiting example, the embodiment 330 includes the substantially plane-parallel screen 310 having circular openings (apertures 314) therethrough having mutually-parallel optical axes and arranged on a square grid with a period slightly exceeding a value of the diameter of the circular openings.

In a related implementation 330 of the optical element 210 of FIG. 2, shown in side view in FIG. 3B, the optical element 330 may include an optical screen 310 and a series of optical components 334 possessing non-zero optical power and defining light paths through the screen 310, such as lenslets with f# of about f/2, for example. Each of the lenslets 334 is bounded by a respectively corresponding opening in the screen 310 and is structured to increase a degree of spatial divergence of light incident onto and traversing the embodiment 330. In one example, the lenslets 334 may include lenslets having negative optical power and adapted to diverge, in transmission through the lenslets, a substantially collimated beam of light at a divergence (half) angle of about 10 to about 40 degrees, preferably within the range of about 10 to about 20 degrees, more preferably about 13 to about 17 degrees, and even more preferably about 15 degrees.

In another implementation (not shown), the optical element 210 includes an optical diffuser such as a holographically-defined LSD Light Shaping Diffuser (available from the Physical Optics Corporation), for example. The surface of such optical diffuser contains substantially randomly distributed, non-periodic surface-relief structures. The optical diffuser for use with an embodiment of the invention is configured to operate in a spectrally-independent fashion, i.e. in white, monochromatic, coherent or incoherent light, by emulating a negative lens in either collimated or non-collimated light without Moire patterns and color diffraction effects. An example of such optical diffuser is provided by the optical component NT 54 500, available from Edmund Scientific, (˜15 degrees of half-angle of divergence, NA˜0.25).

In yet another implementation 350, illustrated schematically in front plan and side views in FIGS. 3C, 3D, the optical transformer element 210 includes a two-dimensional (2D) array of non-zero optical power lenslets 354 at least two of which have non-circular perimeters. The lenslets having non-circular perimeters adjoin each other along their perimeters such as to ensure that areas separating the lenslets 354 from one another are minimized. FIGS. 3C, 3D illustrate but one specific example of a 2D-array of lenslets 354, which has an arbitrarily shaped boundary 356 and in which the adjoining lenslets 354 have rectangular perimeters. However, lenslets shaped differently (for example, lenslets having perimeters defined by closed plane figures having three or more sides such as square perimeters or hexagonal perimeters or perimeters defined by irregularly-shaped closed plane figures) are also within the scope of the invention. Regions separating immediately adjacent lenslets 354 of the embodiment 350 have infinitesimal areas, thereby ensuring that light throughput through the embodiment 350 of the optical transformer element 210 is optimized. In related implementations (not shown), the optical transformer element 210 may include a negative lens collecting substantially all light L delivered from the external source of light, or, alternatively, a diffractive element (for example, a holographically-defined diffraction grating) adapted to transform a substantially collimated beam into an array of optical point sources in far field.

Referring again to the example of FIG. 2, the optical system 216 is configured to have an overall optical magnification that does not depend on the length of the optical system 216. In one example, the optical system includes lenses (as shown, lenses 224, 226) arranged in a telecentric configuration with respect to the optical transformer element 210, such as to image the optical element 210 at an image surface 220 located substantially at an ocular surface (for example, at or near the corneal surface 120). As shown in FIG. 2, for example, the image surface 220 is defined immediately adjacent to and behind, as viewed from the position of the optical element 210, the cornea 120 and in front of an iris 222. In a related example (not shown), the optical system 216 is adapted to ensure that the image surface 220 substantially coincides with the cornea 120. The first component of the optical system 216 (as shown, the lens 224) is preferably separated from the optical element 210 by about a focal length of the first component, and the second component of the optical system 216 (as shown, the lens 226) is separated from the ocular surface of the eye 122 by about a focal length of the second component. Generally, an optical axis of the optical system 216 is parallel an optical axis defined by the transformer element 210. It is appreciated that the overall magnification provided by the optical system 216 does not depend on a separation between a principal plane (not shown) of the optical system 216 and the optical element 210 that is being imaged onto the core. Magnification of the optical system 216 is generally defined by the ratio of the effective focal lengths of the second and first lenses 226, 224. Aggregately, the optical element 210 and the optical system 216 form an optical relay system 230.

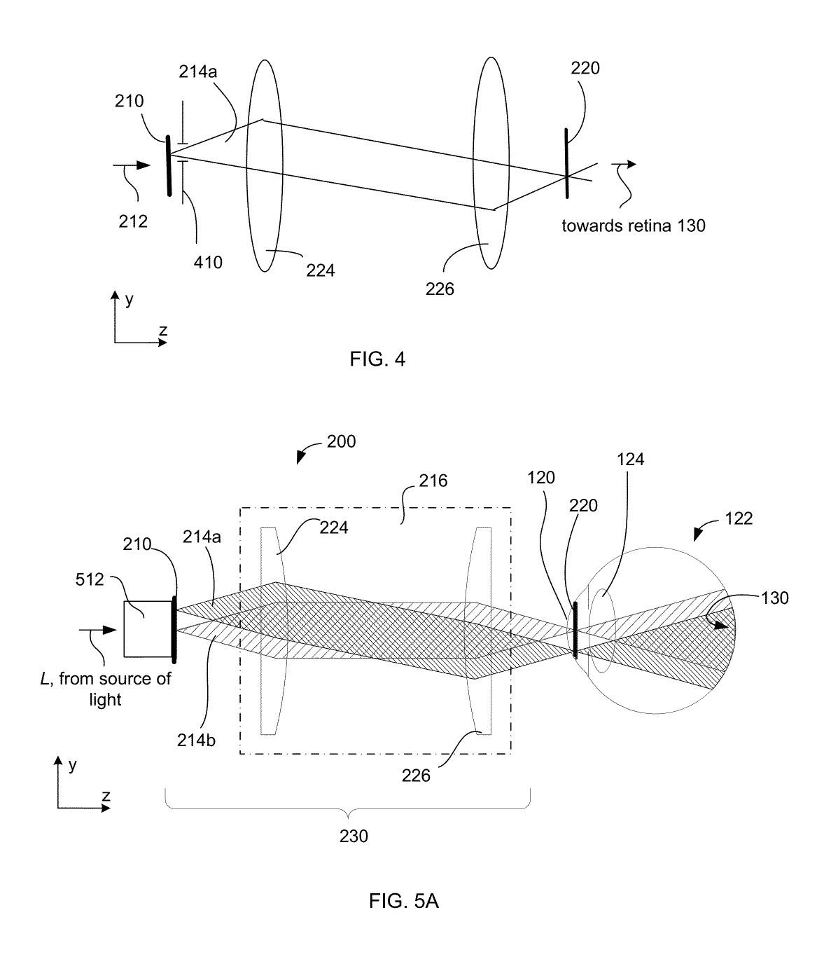

While the optical relay system 230 is shown to include two lenses 224, 226, in various related implementations such relay system 230 may contain a different number of lenses (whether conventional lenses or Fresnel lenses), as well as other optical elements (such as beam-limiting and/or apodizing apertures, prisms, optical filters including interferometric and thin-film filters, and the like) interposed between elements of the system 230. For example, as shown in FIG. 4 that schematically illustrates a portion of the embodiment of FIG. 2, a beam-shaping masking element 410 (as shown, an aperture that may have a spatially-varying transmission profile) is optionally disposed in proximity of the optical element 210 to block a portion of light (in this example, the diverging beam 214b) that forms the image 220 of the optical element 210. In a related implementation (not shown), a beam-shaping element may be disposed between the lenses 224, 226 of the telecentric system 216 or between the outermost lens 226 and the eye 122.

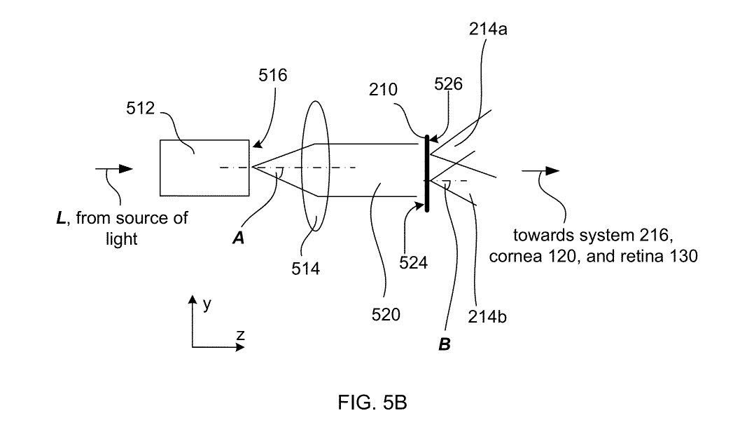

In further reference to FIG. 2, light from a source of light (not shown) can be delivered to the optical transformer element 210 of the embodiment 200 via a fiber optic (FO) component. In one implementation, as shown in FIG. 5A, a FO-element 512 such as a multi-mode optical fiber can be abutted against the optical transformer element 210. Alternatively, as illustrated in FIG. 5B, light L from the source of light (not shown) is delivered to the optical transformer element 210 through the FO-component 512 and an optical collimator 514 (such as a lens, for example) that is spatially articulated with respect to an output facet 516 of the FO-component 512 such as to define a substantially collimated beam 520 illuminating the optical element 210.

In a specific example and in further reference to FIGS. 5B and 2, light is delivered to the optical transformer element 210 in transmission through the FO-component 512 such as multi-mode optical fiber (diameter 0.6 mm, numerical aperture of about NA˜0.4 that corresponds to a half-angle A of divergence of about 23.6 degrees at the output of the FO-component 512) and the lens 514 the numerical aperture of which is preferably matched with that of the FO-component 512. The optical transformer element 210 (which in this example and in reference to FIGS. 3A, 3B includes n=10 light-transmitting apertures or lenslets each of which defines, in transmission, a corresponding beam diverging at an angle B of about 15 degrees (a half-angle value as discussed above). The diverging light that has traversed the element 210 is further incident onto the lenses 224, 226 that form a telecentric system 216 thus defining, in this example, a 1:1 relay system (unity magnification, both angular and linear). The irradiated area of the cornea 120 is about 12 mm in diameter and is separated from the optical element 120 by about 150 mm. The optical system 216 forms, in light converging at about 15 degrees (half-angle), an approximately 12 mm diameter image of the element 210 at the image plane 220 that substantially coincides with the cornea 120. The optical element 210 and the image plane 220 are separated by about 150 mm. The lenses 224, 226 are preferably Fresnel lenses with f-number of about f/0.75 (such as lenses NT43-024, available from Edmund Optics Inc.). A portion 234 of light passing through the cornea, the iris, and the lens of the eye 122 impinges upon the retina 130 as a diverging beam, significantly reducing the irradiance at the retina as compared to the conventional configuration of FIG. 1. In another specific example, the FO-element 512 has a NA of about 0.85 (corresponding to A-58 degrees) and the lens 514 is a 20× objective having f/2 or, alternatively, a 15× objective with f/1.5. It is appreciated that, while the specific parameters of the FO-element 512 and the lens 514 can be varied, their choice ultimately defines the optical throughput at the input 524 of the optical transformer element 210.

Although not discussed specifically, it is appreciated that an embodiment of the invention may include an appropriate housing structure mechanically supporting and/or cooperating at least some of the elements of the embodiment with respect to one another, as well as linear and angular positioners known in the art and adapted to adjust the mutual orientation of the elements of choice. The presence of such housing and/or positioners does not change the principle of the invention. It is also understood that an embodiment of the invention may include a processor programmed, for example, to coordinate the alignment of the optical components of the embodiment, to operate the source of light (which includes changes in the regime of operation of the source of light such as, for example, the light output), and to collect data representing the results of irradiation of an ocular surface (such as the corneal surface) with light from the source of light. For example, in reference to FIGS. 5B and 2, it is envisioned that a portion of light incident from the relay system 230 onto the cornea 120 may be diverted with the use of a beamsplitter and registered by an optical detector to empirically determine the level of irradiance of the cornea.

While the embodiments of the invention (such as those of FIGS. 1B and 2, for example) are shown to operate in transmission of light through at least one of optical components, the skilled artisan will appreciate that a modified embodiment can be easily formed, without undue experimentation, to operate in reflection. In particular, at least one of the elements defining the optical system 164, 216 can include an optical reflector (such as a non-planar mirror element and, in specifically, a curved metallic reflector). Similarly, the optical transformer element 154, 210 can include a reflective surface (for example, transmissive lenslets 354 of the embodiment of FIG. 3C can be replaced with light-diverging reflectors). Generally, therefore, the relay system 230 includes both refractive and reflective optical components. In another modified embodiment, for example, a bundle of optical fibers (such as single-mode optical fibers) can be used to deliver light to the imaging optical system such as the system 216 of FIG. 2. It is understood that in this case the output facets of the individual optical fibers of the bundle are configured to produce an array of diffraction limited local optical sources imaged onto the image surface 220. The output facet of such bundle of optical fibers may optionally be appropriately shaped to ensure that a multiplicity of the output facets lie in a surface that, in a specific case, is imaged by the imaging optical system 216 onto the corneal surface 120.

While the invention has been described through the above-presented examples of embodiments, it will be understood by those of ordinary skill in the art that modifications to, and variations of, the illustrated embodiments may be made without departing from the inventive concepts disclosed herein. Furthermore, disclosed aspects, or portions of these aspects, may be combined in ways not listed above. Accordingly, the invention should not be viewed as being limited to the disclosed embodiment(s).

What is claimed is:

1. A device for illumination of a visual system of a subject, the device comprising:
an optical transformer element configured to transform a substantially collimated distribution of light into a spatially diverging distribution of light; and
an imaging system mechanically cooperated with said optical transformer element to receive a portion of said spatially diverging distribution of light, transmit said portion onto an eye of the subject, and form an image of said optical transformer element at an image surface associated with the eye and located away from a retinal surface of the subject.

2. A device according to claim 1, wherein the optical transformer element includes a holographically-defined light-diffusing element.

3. A device according to claim 1, wherein the optical transformer element includes a diffractive element configured to transform the substantially collimated distribution of light incident onto said diffractive element into an array of optical point sources in far field.

4. A device according to claim 1, wherein the spatially diverging distribution of light includes a plurality of diverging beams of light.

5. A device according to claim 1, wherein the imaging system includes a lens system.

6. A device according to claim 1, wherein the imaging system is configured such as to form an image of said optical transformer element at an ocular surface.

7. A device according to claim 6, wherein a level of irradiance of light at the ocular surface exceeds a level of irradiance of light at the retinal surface.

8. A device according to claim 6, wherein the ocular surface includes a surface of the cornea.

9. A device according to claim 1, wherein the optical transformer element includes at least one of a screen having an array of apertures therein and an array of optical lenslets.

10. A device according to claim 9, wherein first and second optical lenslets from the array of optical lenslets have corresponding non-circular perimeters and adjoin each other along the corresponding non-circular perimeters.

11. A device according to claim 9, wherein a surface of the optical transformer element is substantially planar.

12. A device according to claim 1, wherein an angle of divergence of the spatially diverging distribution of light is about 10 to about 30 degrees.

13. A device according to claim 1, wherein the imaging system is telecentric.

14. A device according to claim 1, further comprising a fiber-optic (FO) element having an input facet adapted to receive light from a source of light and an output facet that is optically cooperated with the optical transformer element to deliver to said optical transformer element a substantially collimated beam of light.

15. A device according to claim 1, further comprising a masking element operably cooperated with a component of the device and configured to block a portion of light that forms the image of the optical transformer element at the image surface.

16. A device according to claim 15, wherein the masking element is positioned proximate to at least one of i) a location of an optical conjugate of an ocular surface as defined by the imaging system and ii) a location of an optical conjugate of the retinal surface as defined by the imaging system.

17. An optical relay system for illumination of a visual system of a subject, the optical relay system comprising:
an optical diffuser configured to transform a beam of light incident onto the optical diffuser to light having a spatially diverging distribution, the optical diffuser having a surface defining a normal to the surface; and
an optical system configured to form an image of said optical diffuser at an image surface located substantially at a corneal surface of the subject when the optical system is positioned for transmitting light from the optical diffuser through the optical system to irradiate the visual system of the subject.

18. An optical relay system according to claim 17, wherein the optical diffuser includes a translucent holographically-defined optical diffuser and the optical system is substantially co-axial with the normal and configured to form an image of the optical diffuser in light transmitted through the optical diffuser.

19. An optical relay system according to claim 17, wherein the optical diffuser includes a diffractive optical element configured to receive a substantially collimated beam of light and form, from such collimated beam of light, a far-field light distribution that includes disconnected spots of light.

20. An optical relay system according to claim 17, further comprising a fiber-optic (FO) element having an input facet adapted to receive light from a source of light and an output facet that is optically cooperated with the optical diffuser such as to deliver a substantially collimated beam of light to the optical diffuser.

21. An optical relay system according to claim 17, further comprising a masking element operably cooperated with a component of the device and configured to block a portion of light that forms the image of the optical diffuser.

22. An optical relay system according to claim 21, wherein the masking element is positioned proximate to at least one of i) a location of an optical conjugate of the corneal surface as defined by the optical system and ii) a location of an optical conjugate of a retinal surface defined in front of the corneal surface.

23. An optical relay system according to claim 17, wherein the optical system is telecentric.

24. A method for illuminating an ocular surface of an eye of a subject, the method comprising:
receiving light from an optical transformer element adapted to transform a substantially collimated beam of light into light having a spatially diverging distribution; and
imaging the optical transformer element with an optical imaging system onto an image surface in front of or behind a retina of the eye such as to transmit a portion of light received from the optical transformer element through a cornea of the eye.

25. A method according to claim 23, wherein said imaging the optical transformer element includes imaging the optical transformer element with an optical imaging system having a magnification that is independent from a distance between a principal plane of the optical imaging system and said optical transformer element.

26. A method according to claim 23, wherein the receiving light from an optical transformer element includes receiving light form an optical transformer element adapted to form, in transmission, a spatial distribution of light having a half angle of divergence between about 10 and about 30 degrees.

27. A method according to claim 23, further comprising at least one of receiving a substantially collimated beam of light at said optical transformer element and transmitting light through said optical transformer element towards a telecentric optical system.

28. A method according to claim 23, wherein said receiving light from an optical transformer element includes receiving light that has traversed an array of optical apertures.

29. A method according to claim 28, wherein said receiving light from an optical transformer element includes receiving light that has traversed an array of optical apertures including first and second apertures, the first and second apertures having non-circular perimeters and adjoining each other along said non-circular perimeters.

30. A method according to claim 28, wherein said receiving light that has traversed an array of optical apertures includes receiving light that has traversed an optical aperture having a non-zero optical power.

31. A method according to claim 28, wherein said receiving light from an optical transformer element includes receiving light from an optical transformer element configured to receive a substantially collimated beam of light and form, from such collimated beam of light, a far-field light distribution that includes an array of point sources of light.

32. A method according to claim 24, further comprising delivering a substantially collimated beam of light to the optical transformer element from a source of light through a fiber-optic (FO) element.

33. A method according to claim 24, further comprising blocking a portion of light, which defines the image of the optical transformer element at the image surface, between the optical transformer element and the cornea.

34. A method according to claim 33, wherein said blocking includes placing a masking element proximate to a location of an optical conjugate of the cornea as defined by the optical imaging system.

35. An optical relay system for illumination of a visual system of a subject, the optical relay system comprising:
an optical diffuser configured to transform a beam of light incident onto the optical diffuser to light having a spatially diverging distribution, the optical diffuser having a surface defining a normal to the surface; and
an optical system configured to irradiate a corneal surface of the subject when the optical system is positioned for transmitting light from the optical diffuser through the optical system to irradiate the retina such that an irradiance value at the corneal surface of the subject exceed that at a retinal surface of the subject.
